(19) United States
(12) Patent Application Publication
(10) Pub. No.: US 2006/0105395 A1
(43) Pub. Date: May 18, 2006

(54) Methods and systems for positioning microspheres for imaging

(76) Inventor: Paul Pempsell, Bedford, TX (US)

Correspondence Address: DAFFER MCDANEIL LLP, P.O. BOX 684908, AUSTIN, TX, 78768, US

(21) Appl. No.: 11/270,786

(22) Filed: Nov. 9, 2005

Related U.S. Application Data
(60) Provisional application No. 60/627,304, filed on Nov. 12, 2004.

(51) Int. Cl. G01N 33/53; G06K 9/00

(52) U.S. Cl. 435/7.1; 382/128

(57) ABSTRACT

Various methods and systems for positioning microspheres for imaging are provided. One system includes a filter medium that includes openings. The openings are spaced in a substantially equidistant manner across the filter medium. The system also includes a flow subsystem coupled to the filter medium. The flow subsystem is configured to exert a force on the microspheres such that the microspheres are positioned above the openings. A method for positioning microspheres for imaging includes exerting a force on the microspheres through a filter medium such that the microspheres are positioned above openings in the filter medium. The openings are spaced as described above.

### PRIORITY CLAIM

This application claims priority to U.S. Provisional Application No. 60/627,304 entitled “Methods and Systems for Positioning Microspheres for Imaging,” filed Nov. 12, 2004, which is incorporated by reference as if fully set forth herein.

### BACKGROUND OF THE INVENTION

1. Field of the Invention

This invention generally relates to methods and systems for positioning microspheres for imaging. Certain embodiments include exerting a force on the microspheres through a filter medium such that the microspheres are positioned above openings in the filter medium. The openings are spaced in an approximately equidistant manner across the filter medium.

2. Description of the Related Art

The following descriptions and examples are not admitted to be prior art by virtue of their inclusion within this section.

Spectroscopic techniques are widely employed in the analysis of chemical and biological systems. Most often, these techniques involve measuring the absorption or emission of electromagnetic radiation by the material of interest. One such application is in the field of microarrays, which is a technology exploited by a large number of disciplines including the combinatorial chemistry and biological assay industries. One company, Luminex Corporation of Austin, Tex., has developed a system in which biological assays are performed on the surface of variously colored fluorescent microspheres. One example of such a system is illustrated in U.S. Pat. No. 5,981,180 to Chandler et al., which is incorporated by reference as if fully set forth herein. In such a fluid flow device, microspheres are interrogated by laser excitation and fluorescence detection of each individual microsphere as it passes at relatively high speed through a detection zone. The measurements of such a system may be easily exported to a database for further analysis.

In the above-mentioned system, fluorescent dyes are absorbed into the microspheres and/or bound to the surface of the microspheres. The dyes are chosen based on their ability to emit light in the wavelength of the chosen detection window. Further, the detection windows are spaced apart by a number of wavelengths, and the dyes are designed to minimize the overlap of a dye's fluorescent signal within adjacent detection windows. By employing two detection windows and two dyes, each at 10 different concentrations, there would thus be 100 fluorescently distinguishable microsphere sets.

One or more biomolecules are also bound to the surface of the microspheres. The one or more biomolecules are selected based on the specific assay to be carried out using the microspheres. For example, one population of microspheres may include different subsets of microspheres, each coupled to a different antigen. The subsets may be combined with a sample, and the assay may be performed to determine which antibodies are present in the sample. The biomolecule(s) that are bound to the microspheres may include any biomolecules known in the art.

The systems described above perform measurements on microspheres while they are flowing through a detection window. The systems provide excellent measurements of the intensity of light scattered by the microspheres and the intensity of light emitted by one or more fluorescent dyes coupled to the microspheres. However, in some instances, it may be desirable to image the microspheres to gain additional or different information about the microspheres and/or a reaction taking or taken place on the surface of the microspheres. Imaging the microspheres as they flow through the systems described above may not be possible due to, for instance, performance limitations of imaging components that are commercially available or economically viable. For instance, the microspheres usually move through an illumination and detection zone at relatively high speeds that limit the time available for imaging of the microspheres. In this manner, images of the microspheres, if formed at all, may have such inferior imaging quality that they do not provide any useful information about the microspheres.

Obviously, therefore, one may attempt to improve the image quality of microsphere images by reducing the speeds at which the microspheres move through the illumination and detection zone thereby increasing the time available for imaging. However, reducing the speeds at which microspheres move through the illumination and detection zone such that imaging may be performed will adversely reduce the throughput of other measurements described above (measurements of scattered light intensity and fluorescent light intensity). In addition, reducing the speeds at which the microspheres move through the illumination and detection zone may not eliminate all obstacles to adequately imaging the microspheres. For example, the solution in which the microspheres are disposed while flowing through the system may adversely affect the image quality.

To form useful images of the microspheres, the microspheres may need to be immobilized in some manner. In addition, the microspheres may need to be immobilized such that the position of the microspheres is sufficiently stable for the length of time necessary to image the microspheres. Although many systems and methods are currently available for immobilizing microspheres, these methods are generally unsuitable for positioning microspheres for imaging. For instance, the materials of some microsphere immobilization systems may prevent adequate illumination of the microspheres for imaging. In addition, the configuration of these microsphere immobilization systems may prevent adequate illumination of the microspheres and collection of light from the microspheres. Furthermore, systems configured to immobilize microspheres for purposes other than imaging will tend to immobilize the microspheres without regard to the spacing between the microspheres. However, suitable spacing between the microspheres may be an important factor in determining whether or not images of the immobilized microspheres can be formed with satisfactory image quality.

Accordingly, it would be advantageous to develop methods and systems for positioning microspheres for imaging that allow sufficient illumination of the immobilized microspheres, sufficient collection of light from the microspheres, and spacing between immobilized microspheres that is suitable for imaging.

### SUMMARY OF THE INVENTION

The following description of various system and method embodiments is not to be construed in any way as limiting the subject matter of the appended claims.

One embodiment relates to a system configured to position microspheres for imaging. The positioning of the microspheres may be performed as a preparation (prep) step before imaging. The system includes a filter medium including openings. The openings are spaced in a substantially equidistant manner across the filter medium. The system also includes a flow subsystem coupled to the filter medium. The flow subsystem is configured to exert a force on the microspheres such that the microspheres are positioned above the openings.

In one embodiment, the flow subsystem is configured to exert the force via suction assisted filtration. In an embodiment, the openings have a diameter that is less than a diameter of the microspheres. In addition, the openings have a diameter that is larger than a diameter of pores of the filter medium. In one embodiment, a number of the openings in the filter medium is approximately equal to a number of the microspheres to be positioned. Alternatively, a number of the openings in the filter medium may be more than or less than the number of the microspheres. The openings may extend through an entire thickness of the filter medium. Alternatively, the openings may extend through a portion of a thickness of the filter medium.

In some embodiments, the system also includes an additional filter medium coupled to the filter medium. In one such embodiment, the flow subsystem is configured to exert the force on the microspheres through the additional filter medium. In one embodiment, the microspheres are in contact with a solution while the microspheres are positioned above the openings. In a different embodiment, the microspheres are not in contact with a solution while the microspheres are positioned above the openings.

In another embodiment, the system includes an imaging subsystem. The imaging subsystem is configured to image the microspheres while the microspheres are positioned above the openings. In one such embodiment, a surface of the filter medium in contact with the microspheres is proximate to an imaging plane of the imaging subsystem. In another such embodiment, a surface of the filter medium in contact with the microspheres is substantially parallel to an imaging plane of the imaging subsystem.

In some embodiments, the imaging subsystem is configured to image the microspheres through the filter medium while the microspheres are positioned above the openings. In another embodiment, the imaging subsystem is configured to image the microspheres with multiple exposures while the microspheres are positioned above the openings. In an additional embodiment, the imaging subsystem includes a charge coupled device (CCD). Alternatively, the imaging subsystem may include any other suitable imaging means or detector known in the art. In a further embodiment, the images generated by the imaging subsystem can be used for bead- or cell-based diagnostic testing. Each of the embodiments of the system described above may be further configured as described herein.

Another embodiment relates to a method for positioning microspheres for imaging. The method includes exerting a force on the microspheres through a filter medium such that the microspheres are positioned above openings in the filter medium. The openings are spaced in an approximately equidistant manner across the filter medium.

In one embodiment, exerting the force is performed using suction assisted filtration. The openings may have a diameter that is less than a diameter of the microspheres. The openings also may have a diameter that is larger than a diameter of pores of the filter medium. A number of the openings in the filter medium may be approximately equal to a number of the microspheres. The openings may extend through an entire thickness of the filter medium. Alternatively, the openings may extend through a portion of a thickness of the filter medium.

Exerting the force on the microspheres may also include exerting the force on the microspheres through an additional filter medium coupled to the filter medium. The microspheres may be in contact with a solution while the microspheres are positioned above the openings. Alternatively, the microspheres may not be in contact with a solution while the microspheres are positioned above the openings.

In some embodiments, the method includes imaging the microspheres while the microspheres are positioned above the openings. In one such embodiment, a surface of the filter medium in contact with the microspheres is proximate to an imaging plane. In another such embodiment, a surface of the filter medium in contact with the microspheres is substantially parallel to an imaging plane.

In some embodiments, the method includes imaging the microspheres through the filter medium while the microspheres are positioned above the openings. In another embodiment, the method includes imaging the microspheres with multiple exposures while the microspheres are positioned above the openings. In an additional embodiment, the method includes imaging the microspheres while the microspheres are positioned above the openings, and images generated by such imaging can be used for bead- or cell-based diagnostic testing. Each of the embodiments described above may include any other step(s) described herein.

### BRIEF DESCRIPTION OF THE DRAWINGS

Other objects and advantages of the invention will become apparent upon reading the following detailed description and upon reference to the accompanying drawings in which:

FIG. 1 is a schematic diagram illustrating a cross-sectional view of a portion of one embodiment of a system configured to position microspheres for imaging;

FIG. 2 is a schematic diagram illustrating a top view of a portion of one embodiment of a system configured to position microspheres for imaging;

FIG. 3 is a schematic diagram illustrating a cross-sectional view of a portion of one embodiment of a system configured to position microspheres for imaging;

FIG. 4 is a schematic diagram illustrating a top view of a portion of one embodiment of a system configured to position microspheres for imaging; and

FIGS. 5-8 are schematic diagrams illustrating a cross-sectional view of a portion of different embodiments of a system configured to position microspheres for imaging.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

The following description generally relates to methods and systems for immobilizing “particles” contained in a solution for the purpose of illumination and/or imaging. The terms “particles” and “particulates” are used interchangeably herein. In addition, the terms “particles” and “microspheres” are used interchangeably herein. The particles may include any discrete substances such as microspheres, cells, or compound aggregates.

According to one method, a solution containing particulates is presented to immobilizing material contained at the bottom of a reservoir suitable for suction assisted filtration (e.g., a filter plate). Once the solution has been filtered through the immobilizing material, and any extra or remaining solution has been removed, the particulates are ready for imaging or illumination.

According to one embodiment, therefore, a system configured to position particles for imaging includes a filter medium and a flow subsystem coupled to the filter medium. The filter medium includes openings. The flow subsystem is configured to exert a force on the microspheres such that the microspheres are positioned above the openings. The flow subsystem may be configured to exert the force via suction assisted filtration.

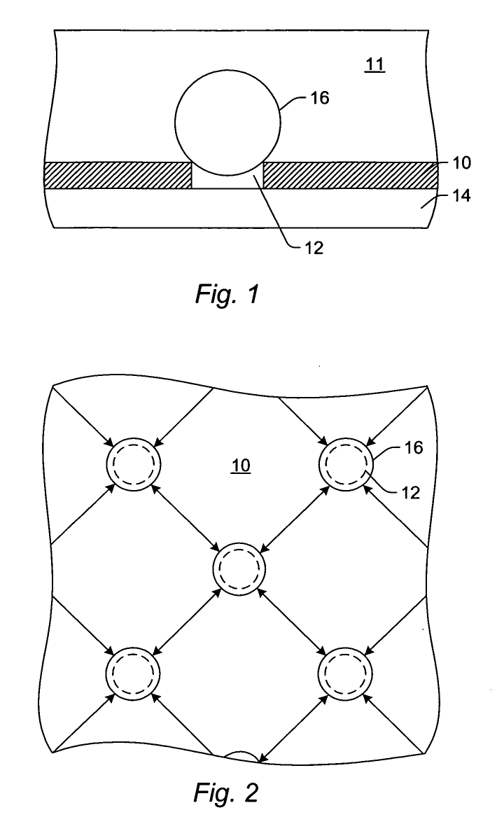

Turning now to the drawings, it is noted that FIGS. 1-8 are not drawn to scale. In particular, the scale of some of the elements of the figures is greatly exaggerated to emphasize characteristics of the elements. It is also noted that FIGS. 1-8 are not drawn to the same scale. Elements shown in more than one figure that may be similarly configured have been indicated using the same reference numerals.

The immobilizing material described herein may include a specially designed perforation pattern in a micro-filter medium. In other words, the specially designed perforation pattern has one or more characteristics such as spacing and lateral dimensions that are different than one or more characteristics of pores in the filter medium. The one or more characteristics of the perforation pattern may be selected based on one or more characteristics of the microspheres and one or more characteristics of an imaging subsystem. For instance, a lateral dimension (e.g., a diameter) of the perforations may be selected based on a lateral dimension (e.g., a diameter) of the microspheres, and a spacing between the perforations may be selected based on one or more characteristics of the imaging subsystem such as angle of incidence and angle of collection. The terms “perforations” and “openings” are used interchangeably herein.

In one embodiment, as shown in FIG. 1, filter medium 10 includes openings 12. The immobilizing material may be constructed by combining two layers of filter sheet material; filter medium 10 and additional filter medium 14 coupled to filter medium 10. Filter media 10 and 14 may be formed of any suitable material or materials known in the art. In addition, filter media 10 and 14 may be formed of the same or different materials. Furthermore, filter media 10 and 14 may have any suitable dimensions.

Filter medium 10 includes perforations that are in direct contact with solution 11, and second filter medium 14 may be un-perforated. These layers will work in conjunction to form wells in which the particles can be substantially immobilized, as shown in FIG. 1. For example, the flow subsystem (not shown in FIG. 1) may be configured to exert a force on microspheres 16 through filter medium 10 and additional filter medium 14 such that microspheres 16 are positioned above openings 12 in filter medium 10. Openings 12 preferably have a diameter that is less than a diameter of microspheres 16. In this manner, microspheres 16 will not completely slip down into the openings and therefore will not be disposed within openings 12 during imaging.

As shown in FIG. 1, openings 12 may extend through an entire thickness of filter medium 10. Alternatively, openings 12 may extend through only a portion of the filter medium. Such openings may be selected, for instance, if additional filter medium 14 is not coupled to filter medium 10. The embodiment of the system shown in FIG. 1 may be further configured as described herein.

The hole-to-hole spacing of the perforation pattern is preferably sufficiently large to allow individual particles to be illuminated and imaged and sufficiently small to allow particles to be included in the flow path of the placement wells. The pattern preferably allows for equidistant particle placement, as shown in FIG. 2. In this manner, as shown in FIG. 2, openings 12 are spaced in a substantially equidistant manner across filter medium 10. Filter media with random particle immobilization wells are currently available. However, such currently available filter media do not facilitate equidistant particulate distribution, which is ideal during particulate imaging.

In one embodiment, a number of the openings in filter medium 10 is approximately equal to a number of the microspheres to be positioned. In this manner, nearly all of the microspheres in a population or sample may be substantially immobilized on filter medium 10 for imaging. In an alternative embodiment, a number of the openings in the filter medium is more than or less than the number of microspheres. In one such embodiment, therefore, not all particles of a population or sample will be positioned on the filter medium. In some instances, a majority of the particles in a population or sample will be positioned on the filter medium.

As shown in FIG. 2, the openings and the microspheres may have a generally circular cross-sectional shape. However, the openings and the microspheres may have any shape known in the art. Therefore, the term “diameter’ as used herein may be replaced with the term “a cross-sectional lateral dimension” if the openings and/or the microspheres have a non-circular cross-sectional shape. The embodiment of the system shown in FIG. 2 may be further configured as described herein.

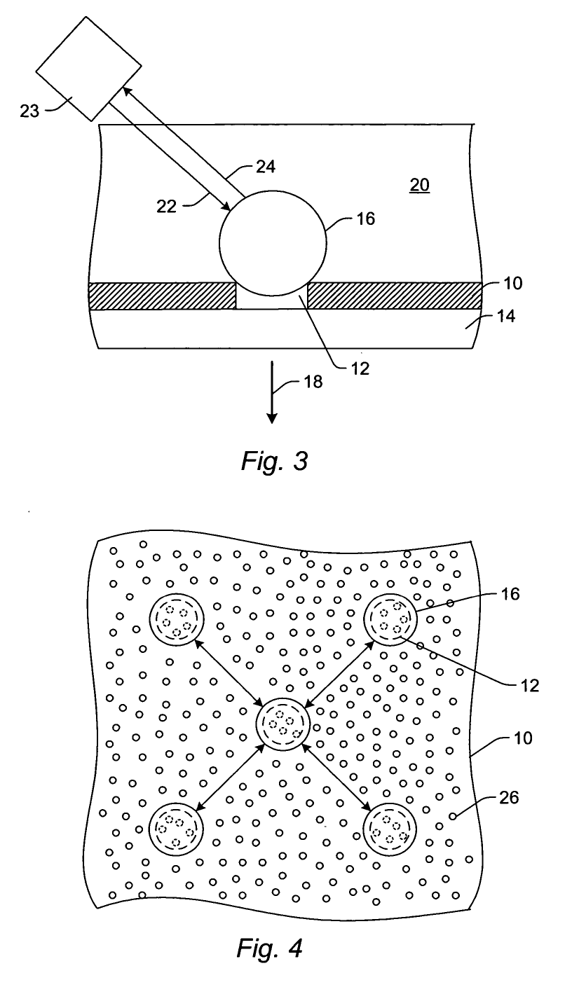

The distance between each opening and therefore between each immobilized microsphere may be selected to allow illumination and imaging of the immobilized microspheres. For example, as shown in FIG. 3, after vacuum 18 is applied to microspheres 16 and solution 20, microspheres 16 will be disposed above openings 12 in filter medium 10. The microspheres are preferably spaced apart such that illumination 22 can be directed to each of the immobilized microspheres by imaging subsystem 23 and such that light 24, returned from the microspheres as a result of the illumination, can be collected and imaged by imaging subsystem 23. Imaging subsystem 23 may be further configured as described herein.

As shown in FIG. 3, therefore, the microspheres may be in contact with solution 20 while microspheres 16 are positioned above the openings. However, the microspheres may not be in contact with solution 20 while microspheres 16 are positioned above openings 12. For example, after immobilization of the microspheres, the solution may be removed as described further herein. Such removal of the solution may be performed if, for example, the solution will interfere with the imaging of the microspheres. It is to be understood, however, that although the solution may be removed, a relatively small amount of solution may be present proximate the microspheres (e.g., a small amount of the solution may be present on the surface of the microspheres).

The illumination may include light having any suitable wavelength known in the art. For example, if a fluorescent image of the microspheres is desired, the wavelength of the illumination may be selected such that the illumination results in the emission of fluorescent light by one or more materials coupled to the microspheres. Alternatively, if a non-fluorescent image of the microspheres is desired, the wavelength of illumination may be selected, for example, to optimize the image quality of the microsphere images. The illumination may also include monochromatic light, near monochromatic light, polychromatic light, broadband light, coherent light, non-coherent light, ultraviolet light, visible light, infrared light, or some combination thereof. As shown in FIG. 3, the illumination may be directed to the microspheres at an oblique angle of illumination. Alternatively, the illumination may be directed to the microspheres at any other suitable angle of illumination (e.g., a normal angle of incidence). The illumination may be provided by a light source (not shown) such as a laser, light emitting diode, or any other suitable light source known in the art.

Light 24 returned from the microspheres as a result of illumination 22 may be collected by one or more optical components (not shown) such as a lens or a mirror. The collected light may be detected by a suitable detector (not shown). For example, the collected light may be detected by a charge coupled device (CCD) or any other imaging means or detector having a two-dimensional array of photosensitive elements (e.g., a time delay integration (TDI) camera). The illumination and the light collection and detection may be performed by imaging subsystem 23 included in the system. In addition to the optical components and configurations described above, imaging subsystem 23 may have any other optical configuration or include any suitable optical components known in the art. The embodiment of the system shown in FIG. 3 may be further configured as described herein.

The holes or perforations are preferably sufficiently larger than the pores of the filter medium, as shown in FIG. 4. In other words, openings 12 have a diameter that is larger than a diameter of pores 26 of filter medium 10. The size of the perforations and the depth of the layer may be selected to immobilize the particles while maintaining sufficient exposure of the particle surface area for illumination or imaging, as shown in FIG. 3. In addition, the pore sizes of the top and bottom filter media layers may be different to optimize the microsphere positioning process.

The particles used in the methods and systems described herein may have a minimum size restriction that correlates to the size of the perforations. For example, the particle size for any given filter medium is preferably large enough such that the immobilized particles are not completely disposed within (do not completely slip down into) the openings, which would complicate illumination and imaging.

Imaging may be performed after the microspheres have been immobilized but while the force (e.g., vacuum) is exerted on the microspheres. Alternatively, the force may be removed from the microspheres if the microspheres will remain relatively stably positioned after the force is removed, and the imaging may then be performed. The embodiment of the system shown in FIG. 4 may be further configured as described herein.

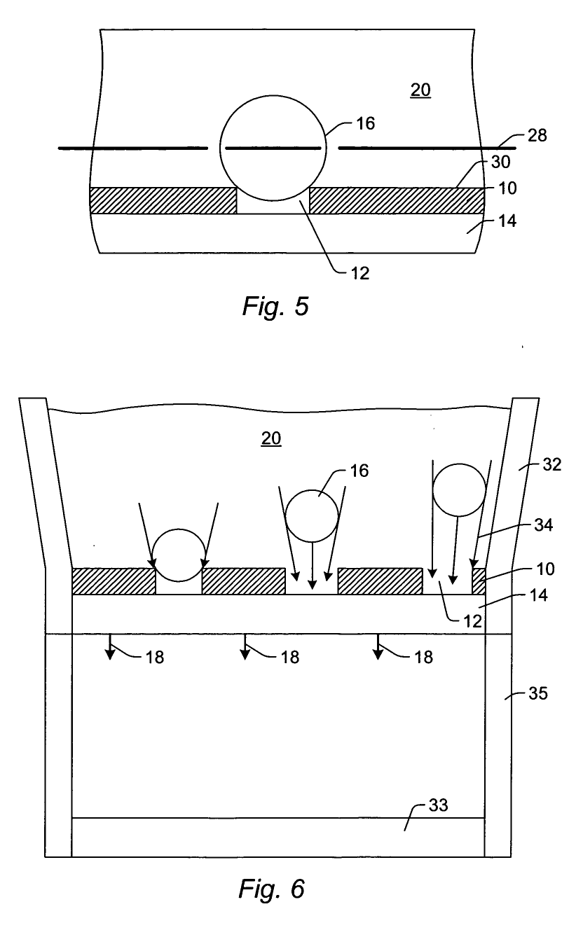

The immobilization of the microspheres creates imaging plane 28, as shown in FIG. 5. The system may also include an imaging subsystem (not shown in FIG. 5), which may be configured as described above. In particular, the imaging subsystem is configured to image the microspheres while the microspheres are positioned above the openings. In this manner, surface 30 of filter medium 10 in contact with the microspheres is proximate to imaging plane 28 of the imaging subsystem. As such, the microspheres will be proximate to the imaging plane of the imaging subsystem. As shown in FIG. 5, the imaging plane of the imaging subsystem may be positioned proximate the center of the microspheres. However, the imaging plane may also be positioned proximate the top of the microspheres or proximate the portion of the microspheres in contact with surface 30 of filter medium 10.

In addition, as shown in FIG. 5, surface 30 of filter medium 10 may be substantially parallel to imaging plane 28 of the imaging subsystem. In this manner, the microspheres will be located at approximately the same position with respect to the imaging plane regardless of their position on the filter medium. As such, the systems and methods described herein will provide adequate focusing of the imaging subsystem across substantially the entire filter medium. Therefore, focus adjustments may be unnecessary between imaging of different microspheres. The embodiment of the system shown in FIG. 5 may be further configured as described herein.

If the immobilizing material is transparent, imaging detection and/or illumination may be performed from either side of the immobilizing material. In other words, an imaging subsystem, which may be configured as described above, may be configured to image the microspheres through the filter medium while the microspheres are positioned above the openings.

In one embodiment, solution 20 containing particulates 16 is presented in reservoir 32 to immobilizing material 10, as shown in FIG. 6. Reservoir 32 may have any suitable configuration known in the art. When vacuum 18 is applied to the bottom of the composite filter media (i.e., the bottom of additional filter medium 14 coupled to filter medium 10), solution fluid flow 34 is created due to the lower restriction in the bottom portion of well 32 (i.e., the portion of well 32 proximate filter medium 10) and applied vacuum 18. Vacuum 18 may be created using flow subsystem 33 coupled to reservoir by conduit 35. Flow subsystem 33 may be configured as described herein. Conduit 35 may include any appropriate conduit known in the art. The particles contained in the solution fluid flow are positioned and immobilized over perforated areas 12 until such a time as a majority of the well areas included in the pattern are populated with particles, as shown in FIG. 6. The system may also include a subsystem (not shown) such as vibrational means configured to facilitate movement of the microspheres into the wells. The embodiment of the system shown in FIG. 6 may be further configured as described herein.

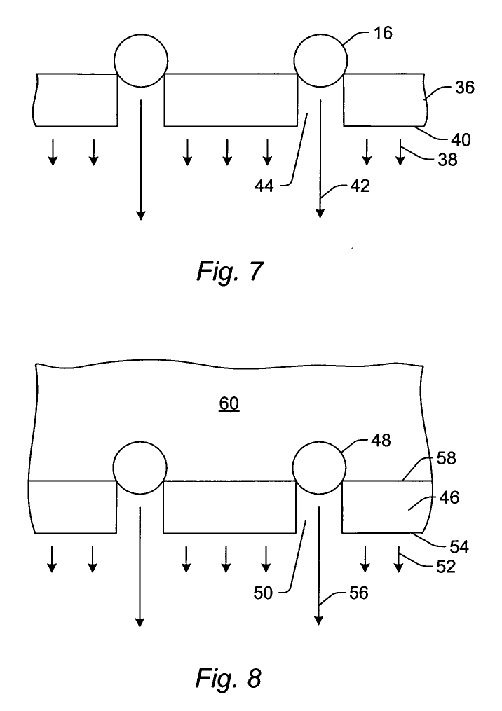

Rather than a double layer of filter media as described above, an alternate immobilizing medium configuration includes a single perforated filter layer or filter medium 36 that can be used to lodge or immobilize particles 16 of a specific size, as shown in FIG. 7. This single layer may be formed of a thicker filter sheet material than that of filter medium 10 to provide adequate mechanical stability to the filter medium. Filter medium 36 may be formed of any suitable material or materials known in the art. Filter medium 36 and openings 44 therein may be formed using any suitable process known in the art. The immobilization of microspheres on filter medium 36 may be performed in a similar manner as described above. For example, vacuum 38 may be applied to one side 40 of filter medium 36 thereby “pulling” solution 42 in which particles 16 were disposed through openings 44 in filter medium 36 and immobilizing particles 16 above openings 44. Openings 44 and filter medium 36 may be further configured as described above. The embodiment of the system shown in FIG. 7 may be further configured as described herein.

Another alternative configuration is perforated solid substrate 46, which may be used to immobilize particles 48 such that the majority of the perforated patterns or openings 50 have been filled with particulates, as shown in FIG. 8. Particles 48 may be immobilized as described above. For instance, vacuum 52 may be applied to side 54 of substrate 46 thereby “pulling” solution 56 through openings 50 and immobilizing particles 48 on side 58 of substrate 46. Solution 60 may be in contact with the immobilized particles. No solution may be drained once the perforations have been “filled” with microspheres. Any remaining solution 60 may be removed by another means, such as those mentioned above. Solid substrate 46 and openings 50 therein may be formed using any suitable materials and processes known in the art. The embodiment of the system shown in FIG. 8 may be further configured as described herein.

Any remaining solution that contains particles may be removed by another means such as siphoning or vacuuming, or the immobilizing material may be rotated to allow any remaining particles to settle outside of the pattern formed in the immobilizing material. Removal of the solution containing non-immobilized particles may be performed with or without maintaining suction on the bottom of the immobilizing material. The number of particulates in the solution may be selected based on the number of perforations in the pattern formed in the immobilizing material. For example, in one embodiment, the filter medium may have a number of openings that is approximately equal to a number of microspheres in the solution.

Imaging particles may not be practical with particles in solution. The particles are preferably placed within a plane at a substantially constant distance from the imaging subsystem or imaging means. Immobilization may be required for long exposure times or multiple exposures. Since the systems and methods described herein provide substantially stable immobilization of microspheres, an imaging subsystem may be configured to image the microspheres with multiple exposures while the microspheres are positioned above the openings since the positions of the microspheres will be substantially stable throughout the extended imaging time needed for multiple exposures. Therefore, the systems and methods described herein may provide more flexibility in the types of microsphere images formed. In addition, multiple exposures may provide more information about the microspheres than a single exposure.

The images of the microspheres may be used for bead- and/or cell-based diagnostic testing, which may include any such testing known in the art. Examples of such diagnostic testing are illustrated in U.S. Pat. No. 5,981,180 to Chandler et al., U.S. Pat. No. 6,046,807 to Chandler, U.S. Pat. No. 6,139,800 to Chandler, U.S. Pat. No. 6,366,354 B1 to Chandler, U.S. Pat. No. 6,411,904 B1 to Chandler, U.S. Pat. No. 6,449,562 B1 to Chandler et al., and U.S. Pat. No. 6,524,793 B1 to Chandler et al., which are incorporated by reference as if fully set forth herein. The assays and experiments in which the microsphere images described herein may be used include any of the assays and experiments described in these patents and any other assays and experiments known in the art.

Another embodiment relates to a method for positioning microspheres for imaging. The method includes exerting a force on the microspheres through a filter medium such that the microspheres are positioned above openings in the filter medium. The openings are spaced in an approximately equidistant manner across the filter medium.

In one embodiment, exerting the force is performed using suction assisted filtration. The openings may have a diameter that is less than a diameter of the microspheres. The openings also may have a diameter that is larger than a diameter of pores of the filter medium. A number of the openings in the filter medium may be approximately equal to a number of the microspheres. The openings may extend through an entire thickness of the filter medium. Alternatively, the openings may extend through a portion of a thickness of the filter medium.

Exerting the force on the microspheres may include exerting the force on the microspheres through an additional filter medium coupled to the filter medium. The microspheres may be in contact with a solution while the microspheres are positioned above the openings. Alternatively, the microspheres may not be in contact with a solution while the microspheres are positioned above the openings.

In some embodiments, the method includes imaging the microspheres while the microspheres are positioned above the openings. In one such embodiment, a surface of the filter medium in contact with the microspheres is proximate to an imaging plane. In another such embodiment, a surface of the filter medium in contact with the microspheres is substantially parallel to an imaging plane.

In some embodiments, the method includes imaging the microspheres through the filter medium while the microspheres are positioned above the openings. In another embodiment, the method includes imaging the microspheres with multiple exposures while the microspheres are positioned above the openings. In an additional embodiment, the method includes imaging the microspheres while the microspheres are positioned above the openings, and images generated by such imaging can be used for bead- or cell-based diagnostic testing. Each of the embodiments described above may include any other step(s) described herein.

It will be appreciated to those skilled in the art having the benefit of this disclosure that this invention is believed to provide methods and systems for positioning microspheres for imaging. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A system configured to position microspheres for imaging, comprising:
a filter medium comprising openings spaced in a substantially equidistant manner across the filter medium; and
a flow subsystem coupled to the filter medium, wherein the flow subsystem is configured to exert a force on the microspheres such that the microspheres are positioned above the openings.

2. The system of claim 1, wherein the flow subsystem is further configured to exert the force via suction assisted filtration.

3. The system of claim 1, wherein the openings have a diameter that is less than a diameter of the microspheres.

4. The system of claim 1, wherein the openings have a diameter that is larger than a diameter of pores of the filter medium.

5. The system of claim 1, wherein a number of the openings in the filter medium is approximately equal to a number of the microspheres to be positioned.

6. The system of claim 1, wherein a number of the openings in the filter medium is more than or less than a number of the microspheres.

7. The system of claim 1, wherein the openings extend through an entire thickness of the filter medium.

8. The system of claim 1, wherein the openings extend through a portion of a thickness of the filter medium.

9. The system of claim 1, further comprising an additional filter medium coupled to the filter medium, wherein the flow subsystem is further configured to exert the force on the microspheres through the additional filter medium.

10. The system of claim 1, wherein the microspheres are in contact with a solution while the microspheres are positioned above the openings.

11. The system of claim 1, wherein the microspheres are not in contact with a solution while the microspheres are positioned above the openings.

12. The system of claim 1, further comprising an imaging subsystem configured to image the microspheres while the microspheres are positioned above the openings, wherein a surface of the filter medium in contact with the microspheres is proximate to an imaging plane of the imaging subsystem.

13. The system of claim 1, further comprising an imaging subsystem configured to image the microspheres while the microspheres are positioned above the openings, wherein a surface of the filter medium in contact with the microspheres is substantially parallel to an imaging plane of the imaging subsystem.

14. The system of claim 1, further comprising an imaging subsystem configured to image the microspheres through the filter medium while the microspheres are positioned above the openings.

15. The system of claim 1, further comprising an imaging subsystem configured to image the microspheres with multiple exposures while the microspheres are positioned above the openings.

16. The system of claim 1, further comprising an imaging subsystem configured to image the microspheres while the microspheres are positioned above the openings, wherein the imaging subsystem comprises a charge coupled device.

17. The system of claim 1, further comprising an imaging subsystem configured to image the microspheres while the microspheres are positioned above the openings, wherein the imaging subsystem comprises an imaging means.

18. The system of claim 1, further comprising an imaging subsystem configured to image the microspheres while the microspheres are positioned above the openings, wherein images generated by the imaging subsystem can be used for bead- or cell-based diagnostic testing.

19. A method for positioning microspheres for imaging, comprising exerting a force on the microspheres through a filter medium such that the microspheres are positioned above openings in the filter medium, wherein the openings are spaced in an approximately equidistant manner across the filter medium.

20. The method of claim 19, wherein said exerting is performed using suction assisted filtration.

21. The method of claim 19, wherein the openings have a diameter that is less than a diameter of the microspheres.

22. The method of claim 19, wherein the openings have a diameter that is larger than a diameter of pores of the filter medium.

23. The method of claim 19, wherein a number of the openings in the filter medium is approximately equal to a number of the microspheres.

24. The method of claim 19, wherein the openings extend through an entire thickness of the filter medium.

25. The method of claim 19, wherein the openings extend through a portion of a thickness of the filter medium.

26. The method of claim 19, wherein said exerting comprises exerting the force on the microspheres through an additional filter medium coupled to the filter medium.

27. The method of claim 19, wherein the microspheres are in contact with a solution while the microspheres are positioned above the openings.

28. The method of claim 19, wherein the microspheres are not in contact with a solution while the microspheres are positioned above the openings.

29. The method of claim 19, further comprising imaging the microspheres while the microspheres are positioned above the openings, wherein a surface of the filter medium in contact with the microspheres is proximate to an imaging plane.

30. The method of claim 19, further comprising imaging the microspheres while the microspheres are positioned above the openings, wherein a surface of the filter medium in contact with the microspheres is substantially parallel to an imaging plane.

31. The method of claim 19, further comprising imaging the microspheres through the filter medium while the microspheres are positioned above the openings.

32. The method of claim 19, further comprising imaging the microspheres with multiple exposures while the microspheres are positioned above the openings.

33. The method of claim 19, further comprising imaging the microspheres while the microspheres are positioned above the openings, wherein images generated by said imaging can be used for bead- or cell-based diagnostic testing.
